(19) United States
(12) Patent Application Publication
(10) Pub. No.: US 2005/0271593 A1
(43) Pub. Date: Dec. 8, 2005

(54) Method for preparation of water-soluble and dispersed iron oxide nanoparticles and application thereof

(75) Inventors: Chen-Sheng Yeh, Tainan City (TW); Fong-Yu Cheng, Kaohsiung County (TW); Dar-Bin Shieh, Tainan City (TW); Chao-Liang Wu, Tainan City (TW)

Correspondence Address: BACON & THOMAS, PLLC, 625 SLATERS LANE, FOURTH FLOOR, ALEXANDRIA, VA, 22314, US

(73) Assignee: National Cheng Kung University, Tainan City (TW)

(21) Appl. No.: 10/882,210

(22) Filed: Jul. 2, 2004

(30) Foreign Application Priority Data
Jul. 31, 2003 (TW) 92121052

(51) Int. Cl. A61K 49/00; C01G 49/02

(52) U.S. Cl. 424/9.36; 423/632

(57) ABSTRACT

The present invention relates to a process for preparing water-soluble and dispersed iron oxide (Fe₃O₄) nanoparticles and application thereof, characterized in which two-stage additions of protective agent and chemical co-precipitation are employed in the process. In the first stage, Fe₃O₄ nanoparticles are obtained using absorbent-reactant coexistence technology. In the second stage, proper amount of adherent is added to cover the nanoparticle surface entirely. The resulting water-soluble and dispersed Fe₃O₄ nanoparticles can easily bind with thiols or biomolecules, such as nucleic acid and peptide. The Fe₃O₄ nanoparticles of the present invention may be used as magnetic resonance imaging contrast agent and used in magnetic guiding related biomolecular technologies for clinical testing, diagnosis and treatment.

### BACKGROUND OF THE INVENTION

1. Field of the Invention

The present invention provides a method for preparing water-soluble and dispersed iron oxide nanoparticles and its applications in magnetic resonance imaging as contrast agent, and in magnetic guiding related biomolecular technologies and clinical testing, diagnosis and treatment.

2. Description of the Related Art

Nanoparticles are very small particles with general size ranging from 1 nm to 100 nm. Given their tiny dimensions, nanoparticles exhibit many special properties related to their surface and volume, for example, very high surface area and surface energy, discrete electronic energy level, special light absorption, and single magnetic domain. Therefore nanoparticles provide great potential in the development of new materials. Every magnetic nanoparticle has specific magnetic orientation. But when the particle is very small, its magnetic field becomes unstable. Such magnetic nanoparticles may be used to carry drug into the body of patients, in which the drug is delivered to different parts of the body through magnetic force. Magnetic nanoparticles can also improve the magnetic resonance imaging (MRI) technology by enhancing the imaging contrast to help doctors identify tumor cells, arterial plaques and central nervous system diseases.

Presently known processes for Fe₃O₄ nanoparticles entail producing uniformly distributed nanoparticles in organic phase. But those nanoparticles exist in aggregate in aqueous solution phase. The coating of nanoparticles with polymer or surfactant helps the nanoparticles to become better dispersed in solution. Most iron oxide nanoparticles used in biomedicine are coated with a layer of substance, such as protein, hydrophilic polymers, starch and glucan, so as to increase their water solubility and dispersibility. When used in intravenous injection, iron oxide nanoparticles coated with hydrophilic substance are 30 to 150 nm in size and mainly in aggregate form.

Contrast agents currently available on the market are mainly Gd³⁺ based. Gadolinium (Gd) is a heavy metal with cytotoxicity. Using improper dosage or formulation of Gd³⁺ contrast agent might produce adverse health effect. Sometimes Gd³⁺ contrast agent produces “false positive signal”, or “false negative signal” when its concentration is diluted by body fluids. Thus the technology that makes use of the super-paramagnetic characteristics of iron oxide nanoparticles for a better and safer contrast agent is worth developing.

### SUMMARY OF THE INVENTION

To address the drawbacks of prior arts for making water-soluble and dispersed iron oxide nanoparticles and the limitation of magnetic resonance imaging contrast agents currently on the market, the present invention discloses a technology for preparing highly water-soluble iron oxide in aqueous phase process that displays super-paramagnetic behavior and may be used as MRI contrast agent. The iron oxide also easily binds biomolecules and drugs due to its simple coating interface and aqueous phase process. Thus the technology disclosed in the present invention may be further developed into a platform technology for functional imaging and target treatment.

An object of the present invention is to provide a method for preparing water-soluble and dispersed Fe₃O₄ nanoparticles, comprising the steps of: (a) mixing solutions containing Fe²⁺ and Fe³⁺ at pre-determined concentration ratio; (b) adding proper amount of organic acid as adherent; (c) adjusting pH value of the foregoing solution to over 10 to produce a precipitate; (d) collecting and washing said precipitate; (e) adding excess amount of organic acid as adherent; (f) adding proper amount of organic solvent and water to remove the excess amount of organic acid in step (e); and (g) collecting purified Fe₃O₄ nanoparticles.

The pre-determined mixing ratio of Fe²⁺ and Fe³⁺ solutions in step (a) is not limited, but preferably 1:2˜1:4 and more preferably 1:2.

The organic acid in steps (b) and (e) is selected from a group consisting of acetic acid, cysteine, alanine, glycine and oleic acid, preferably glycine. The organic acids used in steps (b) and (e) may be the same or different, preferably the same. Said organic acid is used as an adherent. In step (b), Fe₃O₄ nanoparticles are obtained using the adherent-reactant coexistence technology; in step (e), proper amount of adherent is added to achieve complete coating of the nanoparticle surface and result in water-soluble and dispersed Fe₃O₄ nanoparticles.

In step (c) a base, e.g. NaOH, NH₄OH or other similar substances, is added to adjust the pH.

The organic solvent in step (f) is selected from a group consisting of acetone, methanol, ethanol, and n-hexane, preferably acetone.

The aforesaid method is preferably carried out under 20˜40° C., preferably 25° C.

Another object of the present invention is to provide Fe₃O₄ nanoparticles, characterized in which said nanoparticles are water-soluble and well dispersed averaging 6.2 nm±2.2 nm in size.

A further object of the present invention is to provide a contrast agent for magnetic resonance imaging containing primarily water-soluble and dispersed Fe₃O₄ nanoparticles prepared according to the method described above and water.

The method disclosed in this invention can overcome the drawbacks of the currently available technologies to produce uniformly distributed and water-soluble Fe₃O₄ nanoparticles without adding any polymer or surfactant. Such Fe₃O₄ nanoparticles may be used as MRI contrast agent and widely applied in biomedical testing and treatment in the future.

### BRIEF DESCRIPTION OF THE DRAWINGS

FIG. 1 shows the flow chart for preparing Fe₃O₄ nanoparticles according to the invention.

FIG. 2 shows a TEM image of Fe₃O₄ nanoparticles of the invention dissolved in water.

FIG. 3A shows a liver MRI scan prior to using Fe₃O₄ nanoparticle contrast agent.

FIG. 3B shows a liver MRI scan after using Fe₃O₄ nanoparticle contrast agent.

FIG. 4A is a kidney MRI scan prior to using Fe₃O₄ nanoparticle contrast agent.

FIG. 4B is a kidney MRI scan after using Fe₃O₄ nanoparticle contrast agent.

FIG. 5 shows the survival rate of rats after being injected with Fe₃O₄ nanoparticle contrast agent.

### DETAILED DESCRIPTION OF THE INVENTION

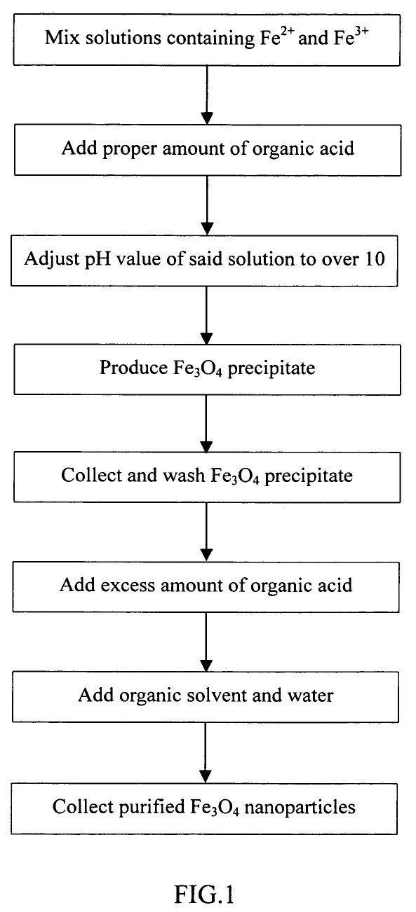

The method for preparing water-soluble and dispersed Fe₃O₄ nanoparticles according to the present invention as shown in FIG. 1 comprises the steps of: mixing solutions containing Fe²⁺ and Fe³⁺ at pre-determined concentration ratio; adding proper amount of organic acid as adherent; (c) adjusting pH value of the foregoing solution to over 10 to produce a precipitate; (d) collecting and washing the precipitate of Fe₃O₄ nanoparticles; (e) adding excess amount of organic acid as adherent; (f) adding proper amount of organic solvent and water to remove the excess amount of organic acid in step (e); and (g) collecting purified Fe₃O₄ nanoparticles.

Examples are illustrated below to depict the preparation of water-soluble and dispersed Fe₃O₄ nanoparticles and its application as MRI contrast agent.

### EXAMPLE 1

### Preparation of Water-Soluble Fe₃O₄ Nanoparticles

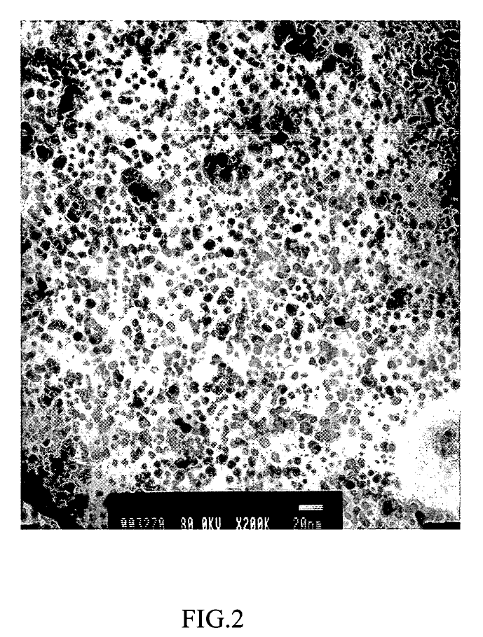

First mix 0.2 M FeCl₂ and 0.1 M FeCl₃ in 2 M HCl solution at the volume ratio of 1:4 (FeCl₂: FeCl₃), then add 1 g of glycine (preferably 0.5˜1.5 g); slowly drip 5 M NaOH solution into the mixture to adjust its pH to over 10 to provide an alkaline environment for Fe₃O₄ in the solution to precipitate; next agitate for 10 minutes, then wash with D.I. water several times to collect the black precipitate (Fe₃O₄); next add 3 g of glycine as adherent; agitate 10‥15 minutes and then vibrate for 30 minutes to let the adherent cover the surface of Fe₃O₄ nanoparticles entirely; subsequently add obtained Fe₃O₄ nanoparticles to acetone and water mixture to remove excess organic acid adherent; centrifuge at 8000 rpm for 20 minutes to precipitate the Fe₃O₄ nanoparticles to obtain water-soluble and dispersed Fe₃O₄ nanoparticles disclosed in the invention. FIG. 2 shows the electron microscope image of resulting Fe₃O₄ nanoparticles dissolved in D. I. water with particle size of 6.2 nm±2.2 nm and exhibiting good, stable and long-lasting water solubility and dispersibility.

### EXAMPLE 2

### Using Fe₃O₄ Nanoparticles as MRI Contrast Agent—Injected in Liver

In this example, Fe₃O₄ nanoparticles prepared in Example 1 were used as MRI contrast agent. The contrast agent was prepared by dissolving the Fe₃O₄ nanoparticles in D. I. water, and if necessary, adding to it proper amount of serum or similar body fluid.

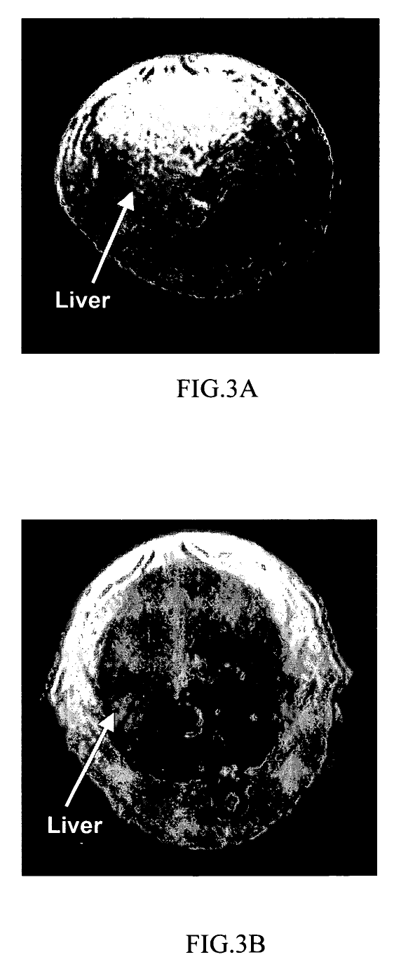

FIG. 3A shows the MRI scan before Fe₃O₄ nanoparticles were injected into the liver; FIG. 3B shows the MRI scan after the liver was injected with 0.86 μM Fe₃O₄ nanoparticles. By comparing where the arrows are pointed at in FIGS. 3A and 3B, it is clearly shown that Fe₃O₄ nanoparticles indeed entered the liver to provide the contrast enhancement effect.

### EXAMPLE 3

### Using Fe₃O₄ Nanoparticles as MRI Contrast Agent—Injected in Kidney

In this example, Fe₃O₄ nanoparticles as described in Example 2 were used as MRI contrast agent and injected in kidney to observe its enhancement effect.

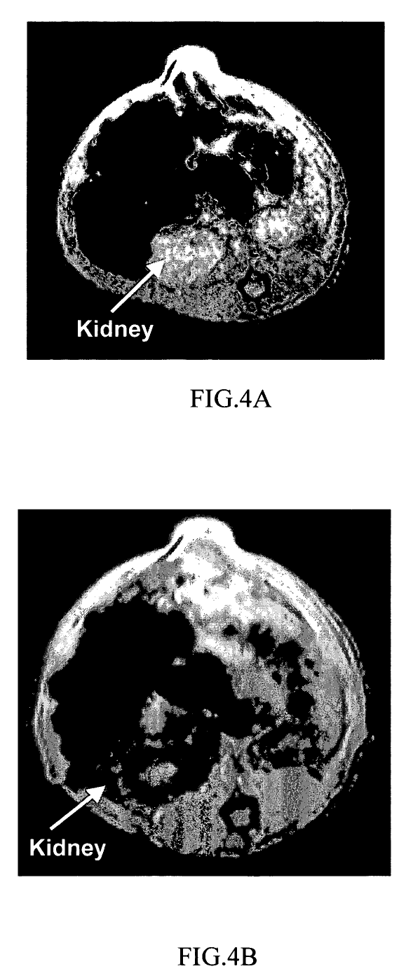

FIG. 4A shows the MRI scan before Fe₃O₄ nanoparticles were injected into the kidney; FIG. 4B shows the MRI scan after the kidney was injected with 0.86 μM Fe₃O₄ nanoparticles. By comparing where the arrows are pointed at in FIGS. 4A and 4B, it is clearly shown that Fe₃O₄ nanoparticles indeed entered the kidney to provide the contrast enhancement effect.

### EXAMPLE 4

### The Safety of Using Fe₃O₄ Nanoparticles as MRI Contrast Agent

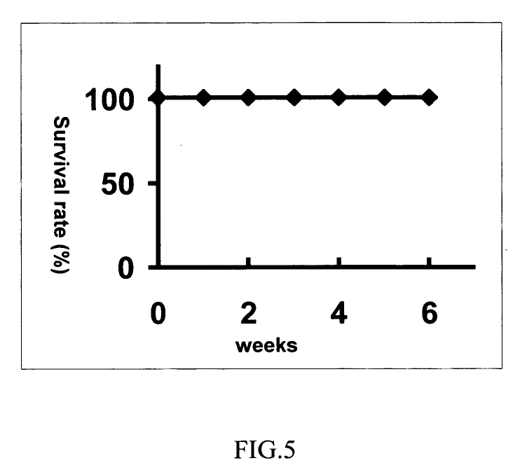

In this test, rats were injected with 5 mg/kg of Fe₃O₄ nanoparticles and observed for survival at week 0, 2, 4, and 6. The finding as shown in FIG. 5 indicates that none of the rates died; the survival rate was 100%. Thus Fe₃O₄ nanoparticles were considered safe as a contrast agent.

To sum up, in comparison with prior art, the technology disclosed herein have the following advantages:
1. The technology disclosed in the invention can produce highly water-soluble and uniformly dispersed Fe₃O₄ nanoparticles without using hydrophilic polymer, surfactant, protein, starch or glucan as protective agent, and offers greater room for subsequent design of surface modification and binding.

2. The Fe₃O₄ nanoparticles of the present invention can bind with nucleic acids, proteins and other biomolecules by forming covalent bond or non-covalent bond for applications in biomedical field.

3. In comparison with contrast agents currently available on the market, the Fe₃O₄ nanoparticle contrast agent herein have very small particle size (6.2 nm±2.2 nm). And because the particle is of nano size and exhibits super-paramagnetic characteristics, its relaxation rate T1 is far lower than the SIPO system on the market (also Fe₃O₄ nanoparticle contrast agent). Table 1 compares the relaxation rate T1 and T2 of Fe₃O₄ nanoparticles herein, SIPO contrast agent, and Gd³⁺ contrast agent.

TABLE 1

| | Fe₃O₄ nanoparticle | | |
|---|---|---|---|
| | contrast agent of the | SIPO | Gd³⁺ contrast |
| | invention | contrast agent | agent |
| T1 | 34 ms | 176 ms | 74.4 ms |
| T2 | 23 ms | 0.77 ms | |

*All contrast agents have the same concentration of 4.61 mM (concentration of metal ion).

4. As shown in Table 1, T1 of the Fe₃O₄ nanoparticles of the invention is much lower than that of SPIO and Gd³⁺ contrast agent. In the aspect of contrast enhancement effect, Gd³⁺ is superior to iron oxide (under ionic concentration of IE-1˜IE-2M). But the Fe₃O₄ nanoparticle contrast agent of the invention exhibits better contrast enhancement effect than SPIO with serum or water as solvent.

5. The T2 of Fe₃O₄ nanoparticle contrast agent of the invention is not lower than SPIO. But the T2 effect of Fe₃O₄ nanoparticle contrast agent of the invention under ionic concentration of 1E-1 1E-2M is comparable to that of SPIO.

6. In comparison with SIPO system available on the market (also iron oxide nanoparticle contrast agent), the Fe₃O₄ nanoparticle contrast agent of the invention is water soluble and dispersed without the protection of starch or glucan. Its T1 effect is better than that of SIPO and its T2 effect is comparable to that of SIPO.

7. In comparison with Gd³⁺ contrast agent, the Fe₃O₄ nanoparticle contrast agent of the invention is non-toxic, has low immunostimulation and does not precipitate in the body. It also costs less to make than the Gd³⁺ process and does not require the protection of chelating agent.

The preferred embodiments of the present invention have been disclosed in the examples. However the examples should not be construed as a limitation on the actual applicable scope of the invention, and as such, all modifications and alterations without departing from the spirits of the invention and appended claims, including the other embodiments shall remain within the protected scope and claims of the invention.

1. A method for preparing water-soluble and dispersed Fe₃O₄ nanoparticles, comprising the steps of:
(a) mixing solutions containing Fe2⁺ and Fe3⁺ at the concentration ratio of 1:2˜1:4;
(b) adding proper amount of organic acid as adherent; said organic acid is selected from a group consisting of acetic acid, cysteine, alanine, glycine and oleic acid;
(c) adjusting pH value of said solution to over 10 to produce a precipitate;
(d) collecting and washing said precipitate;
(e) adding excess amount of organic acid as adherent; said organic acid is selected from a group consisting of acetic acid, cysteine, alanine, glycine and oleic acid;
(f) adding proper amount of organic solvent and water to remove the excess amount of organic acid in the step (e); and
(g) collecting purified Fe₃O₄ nanoparticles.

2. The method according to claim 1, wherein the mixing ratio of solutions containing Fe2⁺ and Fe3⁺ is 1:2.

3. The method according to claim 1, wherein said organic acid is glycine.

4. The method according to claim 1, wherein the organic acids in step (b) and step (e) can be the same or different.

5. The method according to claim 4, wherein the organic acids in step (b) and step (e) are the same.

6. The method according to claim 5, wherein said organic acids are glycine.

7. The method according to claim 1, wherein the precipitate in step (c) is Fe₃O₄.

8. The method accordign to claim 1, wherein the organic solvent in step (f) is selected from a group consisting of acetone, methanol, ethanol and n-hexane.

9. The method according to claim 8, wherein said organic solvent is acetone.

10. The method according to claim 1, wherein the process is carried out under 20˜40° C.

11. The method according to claim 10, wherein the process is carred out under 25° C.

12. The method according to claim 1, wherein the size of Fe₃O₄ nanoparticles is 6.2 nm±2.2 nm.

13. A magnetic resonance imaging contrast agent, comprising water-soluble and dispersed Fe₃O₄ nanoparticles prepared according to claim 1 and water.

14. The magnetic resonance imaging contrast agent accordng to claim 13, wherein the size of Fe₃O₄ nanoparticles is 6.2 nm±2.2 nm.
